(19) United States
(12) Patent Application Publication
(10) Pub. No.: US 2003/0147965 A1
(43) Pub. Date: Aug. 7, 2003

(54) Methods and products useful in the formation and isolation of microparticles

(75) Inventors: Michael Bassett, Providence, RI (US); Jules Jacob, Taunton, MA (US); David Enscore, Sudbury, MA (US)

Correspondence Address: Helen C. Lockhart, Ph.D., Wolf, Greenfield & Sacks, P.C., 600 Atlantic Avenue, Boston, MA, 02210, US

(73) Assignee: Spherics, Inc., Lincoln, RI

(21) Appl. No.: 10/316,128

(22) Filed: Dec. 10, 2002

Related U.S. Application Data
(60) Provisional application No. 60/339,979, filed on Dec. 10, 2001.
(60) Provisional application No. 60/339,980, filed on Dec. 10, 2001.

(51) Int. Cl. A61K 9/16; A61K 9/50; B01J 13/02; B01J 13/04

(52) U.S. Cl. 424/490; 424/491; 424/492; 264/4.1

(57) ABSTRACT

A process for preparing nanoparticles, microparticles and nanoencapsulated products using the PIN process is provided. The invention involves using additives to reduce the aggregation or coalescence of the PIN nanoparticles, microparticles, or nanoencapsulated products during their formation and collection and to facilitate the recovery of said nanoparticles, microparticles, or nanoencapsulated products.

### RELATED APPLICATIONS

This application claims priority under 35 U.S.C. § 119 to U.S. provisional application serial No. 60/339,979, filed Dec. 10, 2001 and to U.S. provisional application serial No. 60/339,980 filed Dec. 10, 2001 each of which is incorporated by reference in its entirety.

### BACKGROUND OF THE INVENTION

Nanoparticles having enhanced drug delivery properties can be prepared by a process referred to as Phase-Inversion Nanoencapsulation (PIN). PIN, as described in U.S. Pat. No. 6,143,211 to Mathiowitz et al., is a process involving conditions which lead to the spontaneous formation of discreet microparticles, including nanospheres. The use of polymers at low concentrations or viscosities, in conjunction with solvent and non-solvent miscible pairs, leads to microparticle formation due to phase inversion of the polymer material when the polymer solution and the non-solvent are rapidly mixed.

The PIN process has many advantages including the ability to incorporate a drug in the microparticles, whether or not the drug is a poorly soluble small organic molecule or a macromolecule (peptide, protein, or DNA). Many different types of polymers are also compatible with the PIN system. For compounds with poor oral bioavailability, use of the PIN system to generate microparticles containing these compounds may facilitate the transfer of the compound across mucosal and/or intestinal barriers. For other compounds, such as protein based drugs, which are characterized by low oral bioavailability due to limited absorption and stability problems under gastric conditions, the PIN system may be used to produce an encapsulated product which protects the drug as well as enhances transport of the drug across the intestinal wall.

The PIN process, however, does have some limitations. For instance, during formation of the PIN product, noticeable aggregation of the primary particles suspended in the non-solvent may occur within 30 seconds of the initial injection of the polymer solution. The reasons for the aggregation may lie in the interaction between the polymer and the non-solvent. This aggregation of primary particles likely causes an increased particle size in the final product upon re-suspension. Since the translocation of PIN particles across the epithelia is size dependent, this aggregation effect can alter overall absorption of the PIN delivery system. Additionally, the particles produced by some versions of the PIN process are small and pliable such that current methods for collection by filtration or centrifugation may fail.

### SUMMARY OF THE INVENTION

The invention, in some aspects, involves methods of producing and collecting particles made using the PIN technology and fabrication process. The methods involve the fabrication of small primary particles, the prevention of particle aggregation, and/or the facilitation of the collection of the PIN particles. The methods of the invention may result in a dramatically improved product yield.

The invention in some aspects provides a method for encapsulating an agent. According to one aspect of the invention, the method involves performing PIN by combining a polymer and an agent in an effective amount of a solvent to form a continuous mixture, and introducing the continuous mixture into an effective amount of a non-solvent containing a dissolved non-solvent soluble polymer to cause the spontaneous formation of a nanoencapsulated product.

Suitable non-solvents include but are not limited to mixtures of isopropyl alcohol and water; mixtures of ethyl alcohol and water; and mixtures of methyl alcohol and water. In one embodiment the non-solvent is 10% to 70% alcohol in water (volume per volume). In one embodiment the non-solvent is 40% to 60% alcohol in water (volume per volume).

Suitable non-solvent soluble polymers include but are not limited to polyvinylpyrrolidone; polyethylene glycol; starch; lecithin; and other natural and synthetic non-solvent soluble polymers or glidants. In some embodiments the concentration of non-solvent soluble polymer in the non-solvent is 0.5% to 10% (weight per volume).

In one embodiment, the non-solvent soluble polymer is polyvinylpyrrolidone and the non-solvent is a mixture of isopropyl alcohol and water.

In some embodiments, the continuous mixture includes an adhesion promoting agent that promotes adhesion of the nanoencapsulated product to a mucosal surface of a body of a subject. Adhesion promoting agents include but are not limited to polyanhydrides and acid anhydride oligomers. In some embodiments, adhesion promoting agents include: iron oxide, calcium oxide, other metal oxides, fumaric acid anhydride oligomers, and poly(fumaric/co-sebacic acid anhydride).

In one aspect of the invention, the non-solvent containing the nanoencapsulated product is spray dried to produce nanoparticles coated with the non-solvent soluble polymer. In one embodiment a solution is added to the nanoparticles coated with non-solvent soluble polymer to produce a suspension. In another embodiment, the nanoparticles coated with non-solvent soluble polymer are compressed to produce a solid oral dosage form.

The agent to be encapsulated may be in a liquid or solid form. It may be dissolved in the solvent, dispersed as solid particles in the solvent, or contained in droplets dispersed in the solvent. One agent of the invention is a bioactive agent. In one embodiment, bioactive agents include, but are not limited to, amino acids, analgesics, anti-anginals, antibacterials, anticoagulants, antifungals, antihyperlipidemics, anti-infectives, anti-inflammatories, antineoplastics, anti-ulceratives, antivirals, bone resorption inhibitors, cardiovascular agents, hormones, peptides, proteins, hypoglycemics, immunomodulators, immunosuppressants, wound healing agents, and nucleic acids.

The nanoencapsulated product of the invention consists of particles having an average particle size between 10 nanometers and 10 micrometers. In some embodiments, the particles have an average particle size between 10 nanometers and 5 micrometers. In yet other embodiments, the particles have an average particle size between 10 nanometers and 2 micrometers, or between 10 nanometers and 1 micrometer or between 10 and 100 nanometers.

The solvent:non-solvent volume ratio may be important in reducing particle aggregation or coalescence. A working range for a solvent:non-solvent volume ratio is between 1:10 and 1:1,000,000. In one embodiment, working range for a solvent:non-solvent volume ratio is 1:10-1:200. In some embodiments, the polymer concentration in the solvent is between 0.1% and 5% (weight per volume).

According to another aspect of the invention, a method for preparing nanoparticles is provided. The method comprises preparing a solution of non-solvent containing a non-solvent soluble polymer and nanoparticles and removing the non-solvent to produce and collect non-solvent soluble polymer coated nanoparticles. Suitable non-solvents include but are not limited to mixtures of isopropyl alcohol and water; mixtures of ethyl alcohol and water; and mixtures of methyl alcohol and water. Suitable non-solvent soluble polymers include but are not limited to polyvinylpyrrolidone; polyethylene glycol; starch; lecithin; modified cellulose and other natural and synthetic non-solvent soluble polymers. In one embodiment, the solvent mixture includes an adhesion promoting agent that promotes adhesion of the polymer-coated nanoparticle to a mucosal surface of a subject. Suitable adhesion promoting agents include but are not limited to polyanhydrides and acid anhydride oligomers. In some embodiments, adhesion promoting agents include: iron oxide, calcium oxide, other metal oxides, fumaric acid anhydride oligomers, and poly(fumaric/co-sebacic acid anhydride).

In one embodiment, the non-solvent soluble polymer is polyvinylpyrrolidone and the non-solvent is a mixture of isopropyl alcohol and water.

The nanoparticles of the invention consists of particles having an average particle size between 10 nanometers and 10 micrometers. In some embodiments, the nanoparticles have an average particle size between 10 nanometers and 5 micrometers. In yet other embodiments, the nanoparticles have an average particle size between 10 nanometers and 2 micrometers, or between 10 nanometers and 1 micrometer or between 10 and 100 nanometers.

In another embodiment of the invention, the method further includes the production of a suspension of an agent by adding a solution to the nanoparticles.

According to yet another aspect of the invention, a suspension of the nanoparticles product is provided. The suspension of nanoparticles product comprises a solution of 0.5% to 10% non-solvent soluble polymer and nanoparticles having an average particle size of less than 10 micrometers. In one embodiment the average particle size of the nanoparticles is less than 1 micrometer. In some embodiments, the nanoparticles include an agent.

The invention also provides a composition of nanoparticles having an average particle size of less than 10 micrometers and coated with a non-solvent soluble polymer. In one embodiment, the average particle size of the nanoparticles is less than 1 micrometer. The nanoparticles composition can be compressed to produce a solid oral dosage form. In one embodiment the nanoparticles composition includes an agent.

According to yet another aspect of the invention, a method for encapsulating an agent is provided. The method involves performing PIN by combining a polymer, an aggregation inhibitor and an agent in an effective amount of a solvent to form a continuous mixture, and introducing the continuous mixture into an effective amount of a non-solvent to cause the spontaneous formation of a nanoencapsulated product.

Suitable polymers include but are not limited to degradable and non-degradable polyesters and include, for example, polylactic acid, polyglycolic acid, and copolymers of lactic and glycolic acid. In some embodiments, the polymer concentration in the solvent phase may be between 0.1% and 5% (weight per volume). In other embodiments, the polymer concentration in the solvent phase may be between 0.1% and 10% (weight per volume).

In one embodiment, the continuous mixture includes an adhesion promoting agent that promotes adhesion of the nanoencapsulated product to a mucosal surface of a subject. Examples of adhesion promoting agents are described above.

The continuous mixture includes an aggregation inhibitor. The aggregation inhibitor may be dissolved or dispersed in the solvent. Aggregation inhibitors include but are not limited to natural and synthetic water-soluble or insoluble polymers. Particularly preferred aggregation inhibitors include: poly(vinylpyrrolidone), poly(ethylene glycol), starch, modified cellulose (i.e., HPMC), and lecithin. In some embodiments, the aggregation inhibitor concentration in the solvent is between 0.01% and 10% (weight per volume).

The agent to be encapsulated may be in a liquid or solid form. It may be dissolved in the solvent, dispersed as solid particles in the solvent, or contained in droplets dispersed in the solvent. One agent of the invention is a bioactive agent. Examples of bioactive agents are described above.

In some embodiments of the invention, the method for encapsulating an agent further comprises freezing the mixture of the solvent, the polymer, the aggregation inhibitor, and the agent to form a frozen mixture, drying to frozen mixture to remove the water, preferably by vacuum. With subsequent drying of the frozen mixture, the dried mixture is then re-dissolved in a solvent prior to addition to the non-solvent. In a preferred embodiment, the mixture of the solvent, the polymer, the aggregation inhibitor, and the agent is frozen in liquid nitrogen.

In some embodiments, the aggregation inhibitor is added to the solvent and to the non-solvent. In one embodiment of the invention, the aggregation inhibitor is added to the solvent and added to non-solvent prior to introduction of the continuous mixture into the non-solvent. In still other embodiments, the aggregation inhibitor is added to the solvent and added to the non-solvent after introduction of the continuous mixture into the non-solvent. In some embodiments, the aggregation inhibitor concentration in the solvent is between 0.01% and 10% (weight per volume) and in the non-solvent is between 0.1% and 20% (weight per volume). In some aspects, the aggregation inhibitor is added only to the non-solvent prior to introduction of the solvent mixture to the non-solvent.

The solvent:non-solvent volume ratio may be important in reducing particle aggregation or coalescence. A working range for the solvent:non-solvent volume ratio is between 1:10 and 1:1,000,000. In one embodiment, working-range for the solvent:non-solvent volume ratio is 1:10-1:200.

The nanoencapsulated product of the invention consists of particles having an average particle size between 10 nanometers and 10 micrometers. In some embodiments, the particles have an average particle size between 10 nanometers and 5 micrometers. In yet other embodiments, the particles have an average particle size between 10 nanometers and 2 micrometers, or between 10 nanometers and 1 micrometer.

According to another aspect of the invention, a method to produce a suspension of an agent by adding a solution to the nanoencapsulated product is provided. The invention also provides a method to produce a solid oral dosage form of the agent comprising compressing the nanoencapsulated product.

According to another aspect of the invention, a method for encapsulating an agent is provided. The method comprises performing phase inversion nanoencapsulation by combining a polymer and an agent in an effective amount of a solvent to form a continuous mixture, and introducing the continuous mixture into an effective amount of a non-solvent to cause the spontaneous formation of a nanoencapsulated product, wherein a water-insoluble aggregation inhibitor is added to the non-solvent. The water-insoluble aggregation inhibitor may be any pharmaceutically acceptable glidant, e.g., talc, kaolin, microcrystalline cellulose, and colloidal silicon dioxide.

In some embodiments of the invention, the water-insoluble aggregation inhibitor is added to the non-solvent prior to the introduction of the continuous mixture into the non-solvent. In other embodiments the water-insoluble aggregation inhibitor is added to the non-solvent after the introduction of the continuous mixture into the non-solvent. The concentration of water-insoluble aggregation inhibitor in the non-solvent is, optionally, between 0.1% and 20% (weight per volume).

In some embodiments, the continuous mixture includes an adhesion promoting agent that promotes adhesion of the nanoencapsulated product to a mucosal surface of a subject. Examples of adhesion promoting agents are described above.

The agent to be encapsulated may be in a liquid or solid form. It may be dissolved in the solvent, dispersed as solid particles in the solvent, or contained in droplets dispersed in the solvent. One agent of the invention is a bioactive agent. In one embodiment, bioactive agents include, but are not limited to, amino acids, analgesics, anti-anginals, antibacterials, anticoagulants, antifungals, antihyperlipidemics, anti-infectives, anti-inflammatories, antineoplastics, anti-ulceratives, antivirals, bone resorption inhibitors, cardiovascular agents, hormones, peptides, proteins, hypoglycemics, immunomodulators, immunosuppressants, wound healing agents, and nucleic acids.

According to another aspect of the invention, nanoparticles and nanoencapsulated products are provided. The nanoparticles and nanoencapsulated products may be produced by the methods of the invention described above.

The invention also encompasses methods for delivering an agent to a subject by administering to the subject a nanoparticle(s) or a nanoencapsulated product including the agent produced according to the methods of the invention.

These and other aspects of the invention, as well as various advantages and utilities, will be more apparent in reference to the following detailed description of the invention. Each of the limitations of the invention can encompass various embodiments of the invention. It is therefore, anticipated that each of the limitation involving any one element or combination of elements can be included in each aspect of the invention.

The foregoing aspects of the invention as well as various objects, features, and advantages are discussed in greater detail below.

### DETAILED DESCRIPTION OF THE INVENTION

The invention in some aspects involves the discovery that the addition of a non-solvent soluble polymer such as polyvinyl pyrrolidone (PVP or PVPD) prevents the aggregation of the microparticles produced during PIN and facilitates the collection of the particles produced by PIN. Thus, the particles produced using this modified version of PIN consistently have a smaller average particle size than particles prepared using the original PIN method and are more efficiently collected. Additionally, these particles have other improved properties such as improved drug solubility.

The method may be performed by combining a polymer and an agent in an effective amount of a solvent to form a continuous mixture, and introducing the mixture into an effective amount of a non-solvent containing a dissolved non-solvent soluble polymer to cause the spontaneous formation of a nanoencapsulated product. This method is a modified form of the PIN method which incorporates the use of non-solvent soluble polymer in the non-solvent to produce very small particles that are capable of being captured and utilized.

Phase inversion nanoencapsulation is a process involving the spontaneous formation of discreet nanoparticles. This one-step process does not require emulsification as a process step. Under proper conditions, low viscosity polymer solutions can be forced to phase invert into fragmented spherical polymer particles when added to appropriate nonsolvents. Phase inversion phenomenon has been applied to produce macro and microporous polymer membranes, hollow fibers, and nano and microparticles forming at low polymer concentrations. PIN has been described by Mathiowitz et al. in U.S. Pat. No. 6,143,211 and U.S. Pat. No. 6,235,224 that are incorporated herein by reference.

PIN is based on a method of “phase inversion” of polymer solutions under certain conditions which brings about the spontaneous formation of discreet nanoparticles. By using relatively low viscosities and/or relatively low polymer concentrations, by using solvent and nonsolvent pairs that are miscible and by using greater than ten fold excess of nonsolvent, a continuous phase of solvent with dissolved polymer can be rapidly introduced into the nonsolvent, thereby causing a phase inversion and the spontaneous formation of discreet microparticles.

Briefly, in the PIN method a polymer is dissolved in an effective amount of a solvent. The agent is also dissolved or dispersed in the effective amount of the solvent. The polymer, the agent and the solvent together form a mixture having a continuous phase, wherein the solvent is the continuous phase. The mixture is introduced into an effective amount of a nonsolvent to cause the spontaneous formation of the microencapsulated product, wherein the solvent and the nonsolvent are miscible and 0<|δ solvent −δ nonsolvent|<6.

These parameters may be adjusted so that the microencapsulated product consists of microparticles having an average particle size of between 10 nanometers and 10 micrometers. The average particle size, of course, may be adjusted within this range, for example to between 50 nanometers and 5 micrometers or between 100 nanometers and 1 micrometer. The viscosity of the polymer/solvent solution also can affect particle size. It preferably is less than 2 centipoise, although higher viscosities such as 3, 4, 6 or even higher centipoise are possible depending upon adjustment of other parameters. It further is possible to influence particle size through the selection of characteristics of the solvent and nonsolvent. For example, hydrophilic solvent/nonsolvent pairs can yield smaller particle size relative to hydrophobic solvent/nonsolvent pairs.

As used herein the terms “nanoparticle” and “nanosphere” are used broadly to refer to particles, spheres or capsules that have sizes on the order of micrometers as well as nanometers. Thus, the terms “microparticle” ”microsphere”, “nanoparticle”, “nanosphere”, “nanocapsule” and “microcapsule” are used interchangeably.

As used herein, a “non-solvent soluble polymer” refers to any suitable material consisting of repeating units including, but not limited to, nonbioerodible and bioerodible polymers that are water soluble. The non-solvent soluble polymer is added to the non-solvent during the PIN process. The traditional PIN process involves the combination of a polymer in a solvent solution with a non-solvent that does not include a polymer. In the methods of the invention non-solvent soluble polymer is added to the non-solvent. Non-solvent soluble polymers include but are not limited to polyvinylpyrrolidone (PVP or PVPD); polyethylene glycol; starch; lecithin; modified celluloses (HPMC, MC, HPC); and other natural and synthetic non-solvent soluble polymers or glidants.

The non-solvent soluble polymer is added to a non-solvent. Suitable non-solvents include but are not limited to mixtures of isopropyl alcohol and water; mixtures of ethyl alcohol and water; and mixtures of methyl alcohol and water. In one embodiment the non-solvent is 10% to 70% alcohol in water (volume per volume). In other embodiments the non-solvent is 20%, 30%, 40%, 50%, 60% 70%, or 80% alcohol in water (volume per volume).

PVP is a preferred non-solvent soluble polymer because it is water soluble. PVP (C₆H₉NO)ₙ(also povidone, polyvidone, poly[1-(2-oxo-1-pyrrolidinyl)ethylene] is a synthetic polymer with a range of molecular weights spanning 2500 to 3,000,000. PVP is most commonly applied to solid dosage forms, where the compound serves as a non-toxic binder in tablets and/or a dissolution enhancing agent for poorly soluble drugs. It is accepted as an excipient in most oral dosing since the compound is not absorbed across intestinal or mucosal surfaces, rendering it non-toxic upon consumption.

The non-solvent soluble polymer can be added to the non-solvent in concentrations ranging from 0.5 to 10% (weight/volume). The non-solvent soluble polymer has not been used in the PIN process for the express purpose of modifying the size of the primary polymer particle itself. The particle size is determined by the operating parameters of the PIN process. In the methods of the invention the non-solvent soluble polymer additive facilitates the collection of the PIN particles.

The non-solvent soluble polymer can be added to the PIN process, allowing the non-solvent soluble polymer /PIN product to be tableted directly or with additional additives into a dosage form. This dosage form can benefit from the binding properties of the non-solvent soluble polymer itself and/or its action as a suspension enhancer upon reconstitution.

In one aspect of the invention, the product produced according to the modified PIN method is spray dried to produce nanoparticles. Spray drying is a method well known in the art. Briefly, in spray drying, the core material to be encapsulated is dispersed or dissolved in a solution. Typically, the solution is aqueous and preferably the solution includes a polymer. The solution or dispersion is pumped through a micrometerizing nozzle driven by a flow of compressed gas, and the resulting aerosol is suspended in a heated cyclone of air, allowing the solvent to evaporate from the microdroplets. The solidified microparticles pass into a second chamber and are trapped in a collection flask.

Although Applicants are not bound by a specific mechanism, it is believed that the non-solvent soluble polymer acts as a particle-forming agent during the spray drying process. Droplets are normally atomized and sprayed into the drying chamber, where the solvent and non-solvent are quickly removed leaving behind the primary particle, which will be lost to waste when the primary particle is small enough. The addition of the non-solvent soluble polymer to the non-solvent will transform the normal droplet into one with a known concentration of non-solvent soluble polymer in it. As the droplet dries, a larger particle can be formed that will contain the smaller primary PIN particle surrounded by the non-solvent soluble polymer. This larger particle may be easily collected, leading to a greater yield of product.

In some aspects of the invention this larger particle can be reconstituted in an aqueous solution. The non-solvent soluble polymer will dissolve, leaving the small particle produced by the PIN process. Additionally the non-solvent soluble polymer dispersed in the aqueous solution will provide an added benefit of a suspension stabilizer.

During the formation of the PIN product using the existing PIN method, noticeable aggregation of the primary particles suspended in the non-solvent may occur within 30 seconds of the initial injection of the polymer solution. The reasons for the aggregation may lie in the interaction between the polymer and the non-solvent. Interaction with the non-solvent is polymer dependent. An example is the interaction between PLGA-based PIN particles and n-heptane. PIN particles composed of 12K PLGA (50:50 L:G) aggregate within 30 seconds of injection, while similar particles based on a 20:80 FA:SA polymer material demonstrate less aggregation. This aggregation of primary particles is the most likely causal factor for an increased size of the particles in the final product upon re-suspension. This particle aggregation may affect overall release or absorption characteristics of the PIN delivery system.

The methods of the invention preserve the primary particle size and also produce microparticles characterized by a homogeneous size distribution making a more accurate and reproducible delivery system. Typical microencapsulation techniques produce heterogeneous size distributions ranging from 10 μm to mm sizes. Prior art methodologies attempt to control particle size by parameters such as stirring rate, temperature, polymer/suspension bath ratio, etc. Such parameters, however, have not resulted in a significant narrowing of size distribution. The PIN method can produce, for example, nanometer sized particles which are relatively monodisperse in size. The modified PIN method of the invention reduces the particle size even further by reducing particle aggregation and accomplishing the capture of particles of very small size. By producing a microparticle that has a well defined and less variable size, the properties of the microparticle such as when used for release of a bioactive agent can be better controlled. Thus, the invention permits improvements in the preparation of sustained release formulations for administration to subjects.

The methods are useful for encapsulating agents. In general, the agents include, but are not limited to, adhesives, gases, pesticides, herbicides, fragrances, antifoulants, dies, salts, oils, inks, cosmetics, catalysts, detergents, curing agents, flavors, foods, fuels, metals, paints, photographic agents, biocides, pigments, plasticizers, propellants and the like. The agent also may be a bioactive agent. The bioactive agent can be, but is not limited to: adrenergic agent, adrenocortical steroid, adrenocortical suppressant, aldosterone antagonist, amino acid, anabolic, analeptic, analgesic, anesthetic, anorectic, anti-acne agent, anti-adrenergic, anti-allergic, anti-amebic, anti-anemic, anti-anginal, anti-arthritic, anti-asthmatic, anti-atherosclerotic, antibacterial, anticholinergic, anticoagulant, anticonvulsant, antidepressant, antidiabetic, antidiarrheal, antidiuretic, anti-emetic, anti-epileptic, antifibrinolytic, antifungal, antihemorrhagic, antihistamine, antihyperlipidemia, antihypertensive, antihypotensive, anti-infective, anti-inflammatory, antimicrobial, antimigraine, antimitotic, antimycotic, antinauseant, antineoplastic, antineutropenic, antiparasitic, antiproliferative, antipsychotic, antirheumatic, antiseborrheic, antisecretory, antispasmodic, antithrombotic, anti-ulcerative, antiviral, appetite suppressant, blood glucose regulator, bone resorption inhibitor, bronchodilator, cardiovascular agent, cholinergic, depressant, diagnostic aid, diuretic, dopaminergic agent, estrogen receptor agonist, fibrinolytic, fluorescent agent, free oxygen radical scavenger, gastrointestinal motility effector, glucocorticoid, hair growth stimulant, hemostatic, histamine H₂ receptor antagonists, hormone, hypocholesterolemic, hypoglycemic, hypolipidemic, hypotensive, imaging agent, immunizing agent, immunomodulator, immunoregulator, immunostimulant, immunosuppressant, keratolytic, LHRH agonist, mood regulator, mucolytic, mydriatic, nasal decongestant, neuromuscular blocking agent, neuroprotective, NMDA antagonist, non-hormonal sterol derivative, plasminogen activator, platelet activating factor antagonist, platelet aggregation inhibitor, psychotropic, radioactive agent, scabicide, sclerosing agent, sedative, sedative-hypnotic, selective adenosine A₁ antagonist, serotonin antagonist, serotonin inhibitor, serotonin receptor antagonist, steroid, thyroid hormone, thyroid inhibitor, thyromimetic, tranquilizer, amyotrophic lateral sclerosis agent, cerebral ischemia agent, Paget's disease agent, unstable angina agent, vasoconstrictor, vasodilator, wound healing agent, xanthine oxidase inhibitor.

Bioactive agents include immunological agents such as allergens (e.g., cat dander, birch pollen, house dust, mite, grass pollen, etc.) and antigens from pathogens such as viruses, bacteria, fungi and parasites. These antigens may be in the form of whole inactivated organisms, peptides, proteins, glycoproteins, carbohydrates or combinations thereof. Specific examples of pharmacological or immunological agents that fall within the above-mentioned categories and that have been approved for human use may be found in the published literature.

The agent to be encapsulated may be in liquid or solid form. It may be dissolved in the solvent or dispersed in the solvent. The agent thus may be contained in microdroplets dispersed in the solvent or may be dispersed as solid microparticles in the solvent or be dissolved in the solvent. The methods of the invention thus can be used to encapsulate a wide variety of agents by including them in either micrometerized solid form or else liquid form in the polymer solution.

The loading range for the agent within the nanoparticles is between 0.01-80% (agent weight/polymer weight). An optimal range is 0.1-50% (weight/weight).

The agent is added to the polymer-solvent mixture, preferably after the polymer is dissolved in the solvent. The solvent is any suitable solvent for dissolving the polymer. Typically the solvent will be a common organic solvent such as a halogenated aliphatic hydrocarbon such as methylene chloride, chloroform and the like, an alcohol, an aromatic hydrocarbon such as toluene, a halogenated aromatic hydrocarbon, an ether such as methyl t-butyl, a cyclic ether such as tetrahydrofuran, ethyl acetate, diethylcarbonate, acetone, or cyclohexane. The solvents may be used alone or in combination. The solvent chosen must be capable of dissolving the polymer, and it is desirable that the solvent be inert with respect to the agent being encapsulated and with respect to the polymer.

The solvent mixture which forms the continuous mixture may include an adhesion promoting agent that promotes adhesion of the nanoencapsulated product to a mucosal surface of a subject (e.g. a human or other mammalian species). Adhesion promoting agents include but are not limited to polyanhydrides and acid anhydride oligomers. Preferred agents are iron oxide, calcium oxide, other metal oxides, fumaric acid anhydride oligimers, and poly(fumaric/co-sebacic acid anhydride).

The method for encapsulating an agent may involve the freezing of the mixture of the solvent, the polymer, and the agent. The freezing step forms a frozen mixture which may be dried using a vacuum. The frozen mixture is then re-dissolved in a solvent prior to addition to the non-solvent. The mixture of the solvent, the polymer, and the agent may be frozen in liquid nitrogen.

The non-solvent is selected based upon its miscibility in the solvent. Thus, the solvent and non-solvent are thought of as “pairs”. The solvent:non-solvent volume ratio may also play a role in reducing particle aggregation or coalescence. A suitable working range for solvent:non-solvent volume ratio is believed to be 1:10-1:1,000,000. An optimal working range for the volume ratios for solvent:non-solvent is believed to be 1:10-1:200 (volume per volume). Such non-solvents include but are not limited to pentane, petroleum ether, hexane, heptane, ethanol, isopropanol/water, mixtures of the foregoing, and oils.

It will be understood by those of ordinary skill in the art that the ranges given above are not absolute, but instead are interrelated. For example, although it is believed that the solvent:non-solvent minimum volume ratio is on the order of 1:10, it is possible that microparticles still might be formed at lower ratios if the polymer concentration is extremely low, the viscosity of the polymer solution is extremely low and the solvent and non-solvent are miscible.

The polymers useful according to the invention for producing the primary PIN particle (and which are dissolved in the solvent) may be any suitable microencapsulation material including, but not limited to, nonbioerodable and bioerodable polymers. Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride and polystyrene.

Examples of preferred non-biodegradable polymers include ethylene vinyl acetate, poly(meth) acrylic acid, polyamides, copolymers and mixtures thereof.

Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as algninate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof. Particularly preferred are polylactic acid, polyglycolic acid, and copolymers of lactic and glycoloic acid.

Preferred polymers are bioadhesive polymers. A bioadhesive polymer is one that binds to mucosal epithelium under normal physiological conditions. Bioadhesion in the gastrointestinal tract proceeds in two stages: (1) viscoelastic deformation at the point of contact of the synthetic material into the mucus substrate, and (2) formation of bonds between the adhesive synthetic material and the mucus or the epithelial cells. In general, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., Van der Waals interactions) and stronger specific interactions, which include hydrogen bonds. The hydrophilic functional groups primarily responsible for forming hydrogen bonds are the hydroxyl and the carboxylic groups. Numerous bioadhesive polymers are discussed in that application. Representative bioadhesive polymers of particular interest include bioerodible hydrogels described by A. S. Sawhney, C. P. Pathak and J. A. Hubell in Macromolecules. 1993, 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly butylmethacrylate), poly(isobutylmethacrylate), poly(hexlmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Most preferred is poly(fumaric-co-sebacic)acid.

Polymers with enhanced bioadhesive properties can be provided wherein anhydride monomers or oligomers are incorporated into the polymer. The oligomer excipients can be blended or incorporated into a wide range of hydrophilic and hydrophobic polymers including proteins, polysaccharides and synthetic biocompatible polymers. Anhydride oligomers may be combined with metal oxide particles to improve bioadhesion even more than with the organic additives alone. The incorporation of oligomer compounds into a wide range of different polymers, which are not normally bioadhesive, dramatically increases their adherence to tissue surfaces such as mucosal membranes.

As used herein, the term “anhydride oligomer” refers to a diacid or polydiacids linked by anhydride bonds, and having carboxy end groups linked to a monoacid such as acetic acid by anhydride bonds. The anhydride oligomers have a molecular weight less than about 5000, typically between about 100 and 5000 daltons, or are defined as including between one to about 20 diacid units linked by anhydride bonds. The anhydride oligomer compounds have high chemical reactivity.

The oligomers can be formed in a reflux reaction of the diacid with excess acetic anhydride. The excess acetic anhydride is evaporated under vacuum, and the resulting oligomer, which is a mixture of species which include between about one to twenty diacid units linked by anhydride bonds, is purified by recrystallizing, for example from toluene or other organic solvents. The oligomer is collected by filtration, and washed, for example, in ethers the reaction produces anhydride oligomers of mono and poly acids with terminal carboxylic acid groups linked to each other by anhydride linkages.

The anhydride oligomer is hydrolytically labile. As analyzed by gel permeation chromatography, the molecular weight may be, for example, on the order of 200-400 for fumaric acid oligomer (FAPP) and 2000-4000 for sebacic acid oligomer (SAPP). The anhydride bonds can be detected by Fourier transform infrared spectroscopy by the characteristic double peak at 1750 cm⁻¹ and 1820 cm⁻¹, with a corresponding disappearance of the carboxylic acid peak normally at 1700 cm⁻¹.

In one embodiment, the oligomers may be made from diacids described for example in U.S. Pat. No. 4,757,128 to Domb et al., U.S. Pat. No. 4,997,904 to Domb, and U.S. Pat. No. 5,175,235 to Domb et al., the disclosures of which are incorporated herein by reference. For example, monomers such as sebacic acid, bis(p-carboxy-phenoxy)propane, isophathalic acid, fumaric acid, maleic acid, adipic acid or dodecanedioic acid may be used.

Organic dyes, because of their electronic charge and hydrophilicity/hydrophobicity, may alter the bioadhesive properties of a variety of polymers when incorporated into the polymer matrix or bound to the surface of the polymer. A partial listing of dyes that affect bioadhesive properties include, but are not limited to: acid fuchsin, alcian blue, alizarin red s, auramine o, azure a and b, Bismarck brown y, brilliant cresyl blue aid, brilliant green, carmine, cibacron blue 3GA, Congo red, cresyl violet acetate, crystal violet, eosin b, eosin y, erythrosin b, fast green fcf, giemsa, hematoylin, indigo carmine, Janus green b, Jenner's stain, malachite green oxalate, methyl blue, methylene blue, methyl green, methyl violet 2b, neutral red, Nile blue a, orange II, orange G, orcein, paraosaniline chloride, phloxine b, pyronin b and y, reactive blue 4 and 72, reactive brown 10, reactive green 5 and 19, reactive red 120, reactive yellow 2, 3, 13 and 86, rose bengal, safranin o, Sudan III and IV, Sudan black B and toluidine blue.

The working molecular weight range for the polymer is on the order of 1 kDa-150,000 kDa, although the optimal range is 2 kDa-50 kDa. The working range of polymer concentration is 0.01-50% (weight/volume), depending primarily upon the molecular weight of the polymer and the resulting viscosity of the polymer solution. In general, the low molecular weight polymers permit usage of a higher concentration of polymer. The preferred concentration range according to the invention will be on the order of 0.1%-10% (weight/volume), while the optimal polymer concentration typically will be below 5%. It has been found that polymer concentrations on the order of 0.1-5% are particularly useful according to the methods of the invention.

Nanospheres and microspheres in the range of 10 nm to 10 μm have been produced according to the methods of the invention. Only a limited number of, microencapsulation techniques can produce particles smaller than 10 micrometers, and those techniques are associated with significant losses of polymer, the material to be encapsulated, or both. This is particularly problematic where the active agent is an expensive entity such as certain medical agents. The present invention provides a method to produce nano to micro-sized particles with minimal losses and can result in product yields greater than 80% and encapsulation efficiencies as high as 100%.

The invention in some other aspects involves the discovery that a class of compounds referred to herein as aggregation inhibitors dramatically improves the properties of microparticles produced using phase inversion nanoencapsulation (PIN). Surprisingly these compounds are capable of reducing the amount of aggregation without impacting the other favorable properties of the particles produced by the PIN method. In some preferred embodiments of the invention, the aggregation inhibitor is used in combination with PLGA, PLA, or FA:SA polymers.

Thus, the particles produced using this modified version of PIN consistently have a smaller average particle size than particles prepared using the original PIN method. Additionally, these particles may have other improved properties such as improved drug solubility.

The method, in some aspects of the invention, may be performed by combining a polymer, an aggregation inhibitor and an agent in an effective amount of a solvent to form a continuous mixture, and introducing the mixture into an effective amount of a non-solvent to cause the spontaneous formation of a nanoencapsulated product. This method is a modified form of the PIN method which incorporates the use of an aggregation inhibitor.

The term “aggregation inhibitor” encompasses “solvent-soluble aggregation inhibitors” as well as “water-insoluble aggregation inhibitors”. As used herein, a “solvent-soluble aggregation inhibitor” refers to a solvent-soluble agent that is an organic solid at room temperature or is of ampiphilic nature and that prevents the aggregation/coalescence of the PIN product during its formation and collection. As used herein, a “water-insoluble” refers to a water-insoluble agent that prevents the aggregation/coalescence of the PIN product during its formation and collection. These compounds are added to and are soluble in the polymer solution phase. Solvent-soluble aggregation inhibitors include, but are not limited to, natural and synthetic water-soluble polymers or glidants, such as polyvinylpyrrolidone (PVP), polyethylene glycol (PEG), starch, and lecithin.

PVP is a preferred solvent-soluble aggregation inhibitor because it is soluble in the polymer solution phase as well as soluble in water, and is thus precipitated when added to the non-solvent phase.

The aggregation inhibitor is added directly to the polymer solution prior to spontaneous particle formation. The aggregation inhibitor can be added in concentrations ranging from 0.1 to 50% of the total polymer content. The existing PIN process allows for a 0.1 to 5% (weight per volume) total polymer concentration in the solvent phase. The aggregation inhibitor prevents the aggregation of these primary particles into larger sized aggregates, which would result in an increased effective particle size. It may be used in the initial polymer solution to maintain the original primary particle size, preventing the typical distribution of PIN material made up of particles and aggregates. The aggregation inhibitor can achieve this by integrating into the polymer particle matrix itself, or by phase-separating and forming a coat around the primary polymer microparticle.

Additional benefits may also be derived from the use of aggregation inhibitors in the formulations using the PIN process. For poorly water-soluble drugs, the aggregation inhibitor coating may have the additional benefit of modifying the release characteristics of the material by enhancing the solubility of the drug. The aggregation inhibitor can be added to the PIN process, allowing the aggregation inhibitor/PIN product to be tableted directly or with additional additives into a dosage form. This dosage form can benefit from the binding properties of the aggregation inhibitor itself and/or its action as a suspension enhancer upon reconstitution.

The methods of the invention also involve the use of a water-insoluble aggregation inhibitor. The method is performed using PIN, but the water-insoluble aggregation inhibitor is added to the non-solvent rather than the polymer solution. The water-insoluble aggregation inhibitors are organic or inorganic molecules in the form of powders with particles that are <100 micrometers, preferably <50 micrometers, and most preferably <25 micrometers in diameter. These agents do not dissolve upon reconstitution of the PIN product in water as does PVP, but, like PVP, are pharmaceutically acceptable additives. They also function to reduce the aggregation of particles during PIN. The PIN method may be performed using a solvent soluble aggregation inhibitor or a solvent insoluble aggregation inhibitor or both.

The water-insoluble aggregation inhibitor can be but is not limited to any pharmaceutically acceptable glidant. Preferred glidants are: talc, kaolin, microcrystalline cellulose, and colloidal silicon dioxide.

In some embodiments of the invention, the water-insoluble aggregation inhibitor is added to the non-solvent prior to the introduction of the solvent mixture into the non-solvent In other embodiments the water-insoluble aggregation inhibitor is added to the non-solvent after the introduction of the solvent mixture into the non-solvent. In either case, the water-insoluble aggregation inhibitor acts within the small time frame between particle formation and the onset of particle aggregation. The concentration of the water-insoluble aggregation inhibitor in the non-solvent is, preferably, between 0.1% and 20% (weight per volume).

The methods of the invention can be, in many cases, carried out in less than five minutes in the entirety. It is typical that preparation time may take anywhere from one minute to several hours, depending on the solubility of the polymer, the solubility of the aggregation inhibitor, and the chosen solvent, and whether the agent will be dissolved or dispersed in the solvent and so on. Nonetheless, the actual encapsulation time typically is less than thirty seconds.

The methods are useful for encapsulating agents examples of which are described above.

In some embodiments of the invention, the method for encapsulating an agent further comprises freezing the mixture of the solvent, the polymer, the solvent soluble aggregation inhibitor, and the agent-containing solution to form a frozen mixture, which is then dried to remove the water, preferably by vacuum. The mixture is then re-dissolved in a solvent prior to addition to the non-solvent. The mixture of the solvent, the polymer, the aggregation inhibitor, and the agent may be frozen in liquid nitrogen.

Because the process does not require emulsification as a process step, it generally speaking may be regarded as a more gentle process than those that require emulsification. As a result, materials such as whole plasmids including genes under the control of promoters can be encapsulated without destruction of the DNA could result from an emulsification process. Thus the invention particularly contemplates encapsulating materials such as plasmids, vectors, external guide sequences for RNAase P, ribozymes and other sensitive oligonucleotides, the structure and function of which could be adversely affected by aggressive emulsification conditions and other parameters typical of certain of the prior art processes.

The invention also provides compositions of the nanoencapsulated products formed by the methods described herein. The nanoencapsulated product or nanoparticles consist of particles having various sizes. In some embodiments the particles have an average particle size of less than 1 micrometer. In other embodiments more than 90% of the particles have a size less than 1 micrometer.

The compositions of the inventions may include a physiologically or pharmaceutically acceptable carrier, excipient, or stabilizer mixed with the nanoparticles. The term “pharmaceutically acceptable” means a non-toxic material that does not interfere with the effectiveness of the biological activity of the active ingredients. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

It is well known to those skilled in the art that microparticles and nanoparticles may be administered to patients using a full range of routes of administration. As an example, nanoparticles may be blended with direct compression or wet compression tableting excipients using standard formulation methods. The resulting granulated masses may then be compressed in molds or dies to form tablets and subsequently administered via the oral route of administration. Alternately nanoparticle granulates may be extruded, spheronized and administered orally as the contents of capsules and caplets. Tablets, capsules and caplets may be film coated to alter dissolution of the delivery system (enteric coating) or target delivery of the nanoparticle to different regions of the gastrointestinal tract. Additionally, nanoparticles may be orally administered as suspensions in aqueous fluids or sugar solutions (syrups) or hydroalcoholic solutions (elixirs) or oils. The nanoparticles may also be administered directly by the oral route without any further processing.

Nanoparticles may be co-mixed with gums and viscous fluids and applied topically for purposes of buccal, rectal or vaginal administration. Microspheres may also be co-mixed with gels and ointments for purposes of topical administration to epidermis for transdermal delivery.

Nanoparticles may also be suspended in non-viscous fluids and nebulized or atomized for administration of the dosage form to nasal membranes. Nanoparticles may also be delivered parenterally by either intravenous, subcutaneous, intramuscular, intrathecal, intravitreal or intradermal routes as sterile suspensions in isotonic fluids.

Finally, nanoparticles may be nebulized and delivered as dry powders in metered-dose inhalers for purposes of inhalation delivery. For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of for use in an inhaler or insufflator may be formulated containing the microparticle and optionally a suitable base such as lactose or starch. Those of skill in the art can readily determine the various parameters and conditions for producing aerosols without resort to undue experimentation. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Techniques for preparing aerosol delivery systems are well known to those of skill in the art. Generally, such systems should utilize components which will not significantly impair the biological properties of the agent in the nanoparticle or microparticle (see, for example, Sciarra and Cutie, “Aerosols,” in Remington's Pharmaceutical Sciences, 18th edition, 1990, pp. 1694-1712; incorporated by reference).

Nanoparticles when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

The compositions are administered to a subject. A “subject” as used herein shall mean a human or vertebrate mammal including but not limited to a dog, cat, horse, cow, pig, sheep, goat, or primate, e.g., monkey.

The compositions are administered in effective amounts. An effective amount of a particular agent will depend on factors such as the type of agent, the purpose for administration, the severity of disease if a disease is being treated etc. The effective amount for any particular application or agent being delivered may vary depending on such factors as the disease or condition being treated, the particular form of the agent being administered, the size of the subject, or the severity of the disease or condition. One of ordinary skill in the art can empirically determine the effective amount of a particular nanoparticle containing agent without necessitating undue experimentation.

Subject doses of the agents encapsulated in the microspheres typically range from about 1 μg to 10,000 mg, more typically from about 10 μg to 5000 mg, and most typically from about 100 μg to 1000 mg. Stated in terms of subject body weight, typical dosages range from about 0.014 μg/Kg to 143 mg/Kg, more typically from about 0.14 μg/Kg to 71 mg/Kg, and most typically from about 1.4 μg/Kg to 14.3 mg/Kg.

Included below are several examples of the methods and the products produced thereby. Although illustrative of the advance in the art achieved by the present invention, it is expected that those skilled in polymer science and microencapsulation processes will, on the basis of these examples, be able to select appropriate polymers, solvents, nonsolvents, solution modifiers, excipients, diluents, encapsulants and so on to spontaneously form microparticles exhibiting desirable properties, including properties desirable for medical applications such as sustained release of bioactive compounds or delivery of drug compounds.

The invention will be more fully understood by reference to the following Examples. These Examples, however, are merely intended to illustrate the embodiments of the invention and are not to be construed to limit the scope of the invention.

### EXAMPLES

### Example 1

Development of PIN Particles using IPA/water Non-Solvent.

1 . 3% PLGA with 25% Isopropyl Alcohol Non-Solvent Phase for Spray Drying

Methods: RG502 PLGA (Boehringer Ingleheim, Petersburg, Va.) was dissolved at 3% (weight/volume) in 60 ml of methylene chloride (EM Science, Gibbstown, N.J.) in a clean vial. In a 4 liter beaker, 3 liters of 25% (volume/volume) isopropanol in water non-solvent was added and agitated via stirplate/stirbar. The polymer solution was quickly added to the isopropanol (EM Science, Gibbstown, N.J.) non-solvent to form the PIN material. The product was then spray-dried via a peristaltic pump into the spray-drying apparatus and collected. Flow input was 10 ml/min inlet temperature was 65° C.

Results: It was difficult to spray-dry and collect the material. It was hypothesized that this was partly due to the high water content in the system. At lower temperatures (less than 50° C.), condensation of materials onto the chamber side walls occurred. Increasing the temperature to 85° C. allowed the material to be spray-dried, but not into small particles.

2. 3% PLGA with 50% Isopropyl Alcohol Non-Solvent Phase for Spray Drying

Methods: Nanoparticles were prepared using a 50% isopropyl alcohol (EM Science, Gibbstown, N.J.) PIN non-solvent containing 2% PVA. (J. T. Baker, Phillipsburg, N.J.) Since the experiment described above used such a high proportion of water, the amount of isopropyl alcohol was increased and the water decreased in this experiment. The polymer (RG502 PLGA) was dissolved in 3% (w/v) in 20 ml of solvent in a clean 20 ml scintillation vial to make a 3% w/v solution. In the GPIN apparatus 1 liter of a 2% PVA (w/v), 50% (v/v) isopropanol in water non-solvent were added to the GPIN process chamber via the injection chamber. The injection valve and the vent valve were open and the filter valve was closed. The polymer solution was added to the injection chamber and the chamber was sealed. The gas was reactivated and the injection valve was quickly opened. The vent valve was closed for 30 seconds. Then the filter valve was opened and the solution was propelled into a clean 4 liter beaker. The beaker was removed and hooked up to a spray drying apparatus. The materials were collected and analyzed for size. 0.6 g of the RG502 PLGA polymer was dissolved in 20 mls of methylene chloride to make a 3% w/v solution. The 50% IPA in water also contained 2% (w/v) polyvinyl alcohol (PVA).

Results: The product was sprayable but it clogged the exit filter of the spray-dryer. Because the dryer compartments operate based on size exclusion, only the smallest particles reach the exit filter. This was indicative that the majority of the particles of the 50% isopropyl alcohol non-solvent were too small to be captured using this procedure.

### Example 2

Development and Isolation of PIN Particles using IPA/water Non-Solvent and Water soluble Polymer to enhance Collection.

1. 3% PLGA with 30% Isopropyl Alcohol and 2% PVP

Methods. RG502 PLGA (Boehringer Ingleheim, Petersburg, Va.) was dissolved at 3% (weight/volume) in 20 ml of methylene chloride (EM Science, Gibbstown, N.J.)in a clean 20 ml scintillation vial. In the GPIN apparatus, 1 liter of a 2% PVP (EM Science, Gibbstown, N.J.) (weight/volume) 30% (volume/volume) isopropanol (EM Science, Gibbstown, N.J.) in water non-solvent was added to the GPIN process chamber via injection chamber with the injection valve and the vent valve open. The filter valve remained closed at this point. The polymer solution was added to the injection chamber and the chamber was sealed. Gas was re-activated and the injection valve was quickly opened. The vent valve was closed and we waited 0.5 minutes. The filter valve was opened and the solution was propelled into a clean 4 liter beaker. The product beaker was removed and hooked up to the spray-drying apparatus. Flow input was 10 mL/min and inlet temperature was 60° C.

Results: The particles prepared by this process were successfully spray dried and captured. By using a 30% IPA non-solvent, a larger particle size was obtained. The larger particle size made the collection steps easier and less particles were lost on the exit filter. The added PVP content facilitated the resuspension and capture of the particles.

2. 3% PLGA with 30% Isopropyl Alcohol Non-Solvent and 2% PVP

Methods: RG502 PLGA was dissolved at 3% (weight/volume) in 20 ml of methylene chloride (EM Science, Gibbstown, N.J.) in a clean 20 ml scintillation vial. In a GPIN apparatus, one liter of a 2% PVP (EM Science, Gibbstown, N.J.) (weight/volume), 30% (volume/volume) isopropanol (EM Science, Gibbstown, N.J.) in water non-solvent was added to the GPIN process chamber via the injection chamber with the injection valve and the vent valve open. Filter valve remained closed at this point. Polymer solution was added to the injection chamber and the chamber was sealed. Gas was re-activated and the injection valve was quickly opened. The vent valve was closed and we waited 0.5 minutes. The filter valve was opened and the solution was propelled into a clean 4 liter beaker. The product beaker was removed and hooked up to the spray-drying apparatus. The materials were spray dried at 50 psi nitrogen feed, flow input of 600 mL/min and inlet temperature of 65 ° C.

Results: The experiment resulted in the successful collection of the majority of the pin product without clogging the exit filter. Particle sizing was performed using 15 mg of sample in 3 mls of a 0.1% SDS with 0.03% sodium azide solution. Samples were sonicated for 2 minutes in a bath sonicator and run. The sample parameters and resulting data is also shown in Table I.

3. 3% PLGA with 50% Isopropyl Alcohol Containing 2% PVP, with Low Pressure Spray Drying.

Methods: 3% (w/v) RG502 PLGA was dissolved in 20 ml of methylene chloride (EM Science, Gibbstown, N.J.) in a clean 20 ml scintillation vile. In the GPIN apparatus, 1 liter of a 2.0% PVP (EM Science, Gibbstown, N.J.) (w/v), 50% (v/v) isopropanol (EM Science, Gibbstown, N.J.) in water non-solvent was added to the GPIN process chamber through the injection chamber. The injection valve and vent valve were open and the filter valve was closed. The polymer solution was added to the injection chamber and the chamber was sealed. The gas was reactivated and the injection valve was quickly opened. The vent valve was closed for 0.5 minutes. Then the filter valve was opened and the solution was propelled into a clean 4 liter beaker. The beaker was removed and hooked up to a spray drying apparatus. The inlet pressure of the spray dryer was reduced from 50 to 10 psi to enlarge the incoming droplet size. The purpose of doing this was to produce a larger droplet which will enhance the collection of even smaller particles.

Results: The experiment yielded unexpected results. Dramatic recovery of small particles was accomplished. Particle sizing using a 50 micrometer aperture demonstrated the collection of particles in which 90% were less than 2 micrometers in number diameter (number average diameter—Dia (N) )and had a volume diameter (volume average diameter—Dia (V) ) of less than 3.2 micrometers. The data is shown in Table I.

4. 3% PLGA in 50% IPA with 0.18% PVP

Methods: The methods were performed as described above in number 3, but 0.18% of PVP was used.

Results: This method resulted in the collection of small microparticles.

TABLE I

| Batch | Number Diameter 90%< | Volume Diameter 90%< |
|---|---|---|
| Example 2.2 | 0.782 | 1.567 |
| Example 2.2 | 0.795 | 1.499 |
| Example 2.2 | 0.807 | 1.522 |
| Example 2.3 | 0.995 | 1.915 |
| Example 2.3 | 0.972 | 1.832 |
| Example 2.3 | 0.97 | 1.991 |
| Example 2.4 | 1.32 | 2.781 |
| Example 2.4 | 1.314 | 2.787 |
| Example 2.4 | 1.303 | 2.69 |

Example 3: Preparation of microparticles using PVP

The following experiment was performed in order to demonstrate the effects of the addition of 10% PVP in a polymer solution during PIN on the resuspension and particle size distribution of the microparticle product.

Methods: Several batches of microparticles were prepared using the following procedure. Polymer was dissolved in 20 ml of methylene chloride (DCM) in a clean 20 ml scintillation vial at a 3% (w/v) concentration. In the GPIN (generic phase inversion nanoencapsulation) apparatus, 1000 ml of heptane was added to the GPIN process chamber via the injection chamber, with the injection valve and vent valve open. The filter valve was left closed at this point. One Whatman 50 filter was placed in the millipore filter apparatus and sealed with hex-bolts. The chamber was swept with nitrogen and then the gas was shut off and the injection valve was closed. The polymer:DCM solution was added to the injection chamber an the chamber was sealed. The gas was reactivated and the injection valve was quickly opened. The vent valve was closed for 0.5 minutes and then the filter valve was opened and the solution was propelled through the millipore filter apparatus with gas pressure set to 2-3 psi. The system was continuously flushed with nitrogen for 2 minutes to dry the particles to the filter. After this time, the gas supply was stopped and the filter with the PIN particles was carefully removed. The PIN particles were removed from the paper into a pre-weighed clean 20 ml scintillation vial in the presence of a Plas Labs Pulse Ionizer (serial no. 55228), (VWR, Bridgeport, N.J.) to inhibit static behavior. The top of the vial was covered with perforated foil, and the particles were subjected to size analysis.

The following materials were used in the microparticle preparation process:

Polymer: RG502PLGA 50:50-Boehringer Ingleheim-(Petersburg, Va.)

PVP: EM Science, OMNIPURE, polyvinyl pyrrolidone, (VWR, Bridgeport, N.J.)

MeCL₂: EM Science, dichloromethane, Omnisolv, (VWR, Bridgeport, N.J.)

N-heptane: J. T. Baker, ultra resi-analyzed, (VWR, Bridgeport, N.J.)

The polymer and PVPD were dissolved in 20 ml MeCL₂. It was this solution which was added to 1000 ml N-heptane in the PIN chamber.

The following proportions of materials were used in the experiments:

Form. 1. 1%: 6.0 mg PVP plus 594 mg RG502

The weight of the filter paper before the experiment was 590.0 mg and after the experiment was 1168.2 mg. The weight of the recovered PIN product was 5715 mg.

Form. 2. 5%: 30.1 mg PVP plus 570 mg RG502.

The weight of the filter before the experiment was 596.5 mg and after the experiment was 1169.1 mg. The weight of the recovered PIN product was 565.3 mg.

Form. 3. 15%: 40.1 mg PVP plus 510 mg RG502.

The weight of the filter paper before the experiment was 589.9 mg and after the experiment was 1145.0 mg. The weight of the recovered PIN product was not measured.

Form. 4. 25%: 150.1 mg PVP plus 450 mg RG502

The weight of the filter paper before the experiment was 596.5 mg and after the experiment was 1177.8 mg. The weight of the recovered PIN product was 568.3 mg.

Form. 5. 50%: 300.0 mg PVP plus 300.1 mg RG502

The weight of the filter paper before the experiment was 596.0 mg and after the experiment was 1184.6 mg. The weight of the recovered PIN product was 579.4 mg.

The PVP PIN products prepared according to these specifications were examined using a Beckman Coulter Multisizer III with a 50 micrometer aperture in order to determine the size of the particles. The samples were resuspended in 2 ml 0.1% sodium lauryl sulfate (SLS) (VWR, Bridgeport, N.J.) in distilled water via a 3 minute bath sonication.

Results: Samples of microparticles were prepared using the PIN methodology and differing amounts of PVP as described above. These microparticles were examined to determine the average particle size using a Beckman Coulter Multisizer III. Table II presented below lists the amount of microparticle sample tested and the average particle size.

TABLE II

| Example 3 | Mass | Dia(V) | Avg D | Dia(N) | Avg |
|---|---|---|---|---|---|
| Formulation # | (mg) | (90%) | (V) | (90%) | D(N) |
| Form. 1 | 4.8 | 3.14 | 2.85 | 2.08 | 1.76 |
| Form. 1 | 5.4 | 2.56 | | 1.437 | |
| Form. 2 | 4.5 | 2.988 | 2.824 | 1.530 | 1.529 |
| Form. 2 | 4.5 | 2.661 | | 1.531 | |
| Form. 3 | 5.9 | 2.347 | 2.305 | 1.543 | 1.541 |
| Form. 3 | 6.5 | 2.264 | | 1.539 | |
| Form. 4 | 7.2 | 2.674 | 2.756 | 1.654 | 1.650 |
| Form. 4 | 7.1 | 2.839 | | 1.645 | |
| Form. 5 | 10.5 | 3.081 | 3.240 | 1.858 | 1.903 |
| Form. 5 | 9.7 | 3.398 | | 1.948 | |

The PVP PIN microparticle samples were also analyzed for size on the Beckman Coulter Multisizer III with a 20 micrometer aperture. The samples were resuspended in 2 ml of the 0.1% SLS resuspension buffer with a 3 minute bath sonication. The results of the size analysis are shown in Table III below.

TABLE III

| Batch | Form. 1 | Form. 2 | Form. 3 | Form. 4 | Form. 5 |
|---|---|---|---|---|---|
| Dia(N) | 0.895 | 0.89 | 0.906 | 0.958 | 1.153 |
| 90%< | 0.882 | 0.884 | 0.886 | 0.99 | 1.122 |
| Average | 0.8885 | 0.887 | 0.896 | 0.974 | 1.1375 |
| Dia(V) | 1.357 | 1.348 | 1.269 | 1.585 | 3.441 |
| 90%< | 1.235 | 1.337 | 1.307 | 1.819 | 2.936 |
| Average | 1.296 | 1.3425 | 1.288 | 1.702 | 3.1885 |

Some of the samples were resized after 5-6 hours with and without a 1 minute sonication) The results of the analysis are listed in Table IV.

TABLE IV

| Batch | Form. 1 | Form. 2 | Form. 3 | Form. 4 | Form. 5 |
|---|---|---|---|---|---|
| Dia(N) | 0.839 | 0.872 | 0.866 | 0.920 | 1.023 |
| 90%< | | | | | |
| Dia(V) | 1.042 | 1.150 | 1.114 | 1.310 | 1.813 |
| 90%< | | | | | |
| | Without | Without | Without | Without | Without |
| | sonication | sonication | sonication | sonication | sonication |
| Dia(N) | 0.881 | 0.902 | 0.906 | 0.976 | 1.209 |
| 90%< | | | | | |
| Dia(V) | 1.291 | 1.429 | 1.282 | 1.770 | 3.756 |
| 90%< | | | | | |
| | With a 1 minute | With a 1 minute | With a 1 minute | With a 1 minute | With a 1 minute |
| | sonication | sonication | sonication | sonication | sonication |

### Example 4

Preparation of PVP Containing Microparticles with Insulin

The purpose of the experiment was to prepare microparticles containing insulin using the PVP technology described in Example 3.

Materials and Methods: The following materials were used in the process: RG502 PLGA (Boehringer Ingleheim (Petersburg, Va.)), FAPP (Spherics Incorporated, Warwick, R.I.), Fe₃O₄ (Fisher Scientificunknown lot no. 854319), PVP ((VWR, Bridgeport, N.J.), EM), petroleum ether ((VWR, Bridgeport, N.J.), EM), DCM ((VWR, Bridgeport, N.J.), EM ), micro tBA insulin (Spherics, Warwick, R.I.,).

In each of the experiments described below, polymer was dissolved in 20 ml of methylene chloride (DCM) in a clean 20 ml scintillation vial at a 3% (w/v) concentration, or 600 mg, 90 mg FAPP, 60 mg PVP and 60 mg Fe₃O₄. The appropriate amount of insulin was added to this mixture. In a clean 1 liter beaker 1000 ml of n-heptane was added to the mixture. The insulin suspension was sonicated for 1 minute, and then quickly added to the petroleum ether, which was stirred with a spatula. The resultant product was filtered through a Buchner funnel containing a 1 micrometer filter. The PIN product was removed from the paper into a clean 20 ml scintillation vial in the presence of the PLAS Labs Pulse Ionizer (serial no. 5528) to inhibit static behavior. The top of the vial was covered with a perforated foil and placed on a manifold freeze-drier.

Two particle preparations were prepared, one with a 10% final insulin concentration (w/w) or 90 mg, and the other a 5% final (w/w) concentration or 42.7 mg.

Each formulation was dissolved in 20 mls of DCM and sonicated for 1 minute in a bath sonicator. The solution was immediately added to 1 liter of petroleum ether and stirred with a spatula and filtered through a 1 micrometer filter. The product was collected in a 20 cc vial and freeze-dried.

The results of the particle size analysis of these products is shown in Table V.

TABLE V

| | Dia (N) | Dia (N) | Dia (V) | Dia (V) |
|---|---|---|---|---|
| Sample | Mean (μm) | %<90 (μm) | mean (μm) | %<90 (μm) |
| Form 1, 5% | 1.608 | 2.287 | 2.415 | 4.843 |
| | 1.595 | 2.183 | 2.259 | 4.083 |
| Form 2, 10% | 1.500 | 1.917 | 1.836 | 2.922 |
| | 1.457 | 1.830 | 1.795 | 2.872 |

As shown in the above table, the particles prepared using the PIN method with PVP resulted in significantly reduced particle size compared to those prepared by the PIN process without PVP (Example 5).

### Example 5

Preparation of PIN using no PVP Additive, a Control Study

The purpose of this study was to produce PIN batches using the process outlined herein. This study produced PIN without the use of PVP as an aggregation inhibitor.

Materials and methods: The following materials were used in the process: RG502 PLGA (Boehringer Ingleheim, Petersburg, Va.), methylene chloride (EM Science, VWR, Bridgeport, N.J.), petroleum ether (J. T. Baker, VWR, Bridgeport, N.J.).

In the experiment described below, 300 mg of RG502 PLGA was dissolved in 10 ml of methylene chloride. In a clean vessel, 500 ml of petroleum ether was added. The polymer solution was quickly added to the non-solvent petroleum ether and swirled. The product was filtered and then collected inot a clean scintillation vial in the presence of a Plas Labs Pulse ionizer (VWR, Bridgeport, N.J.). The product was partially covered and set to dry on the manifold freeze dryer.

The product was submitted for particle size analysis. The results are given in Table VI below.

TABLE VI

| | Dia (N) | Dia (N) | Dia (V) | Dia (V) |
|---|---|---|---|---|
| Sample | Mean (μm) | %<90 (μm) | mean (μm) | %<90 (μm) |
| Example 5 | 1.601 | 2.209 | 2.309 | 4.575 |
| Control Study | 1.589 | 2.173 | 2.370 | 4.758 |
| | 1.608 | 2.201 | 2.368 | 4.776 |

We claim:

1. A method for encapsulating an agent, comprising:
performing phase inversion nanoencapsulation by combining a polymer and an agent in an effective amount of solvent to form a continuous mixture, and introducing the mixture into an effective amount of a non-solvent containing a dissolved non-solvent soluble polymer to cause the spontaneous formation of a nanoencapsulated product.

2. The method of claim 1 wherein the non-solvent is selected from the group consisting of: mixtures of isopropyl alcohol and water; mixtures of ethyl alcohol and water; and mixtures of methyl alcohol and water.

3. The method of claim I wherein the non-solvent soluble polymer is selected from the group consisting of: polyvinylpyrrolidone; polyethylene glycol; starch; lecithin; modified cellulose; and other natural and synthetic water-soluble polymers or glidants.

4. The method of claim 1 wherein the non-solvent soluble polymer is polyvinylpyrrolidone and the non-solvent is a mixture of isopropyl alcohol and water.

5. The method of claim 1 wherein the continuous mixture further comprises an adhesion promoting agent that promotes adhesion of the nanoencapsulated product to a mucosal surface of a subject.

6. The method of claim 5 wherein the adhesion promoting agent is chosen from the group consisting of: iron oxide; calcium oxide; other metal oxides; fumaric acid anhydride oligimers; poly(fumaric/co-sebacic acid anhydride); and other polyanhydrides and acid anhydride oligimers.

7. The method of claim 2 wherein the non-solvent is 10% to 70% alcohol in water (volume per volume).

8. The method of claim 2 wherein the non-solvent is 40% to 60% alcohol in water (volume per volume).

9. The method of claim 1 wherein the concentration of non-solvent soluble polymer in the non-solvent is 0.5% to 10% (weight per volume).

10. The method of claim 1 wherein the non-solvent containing the nanoencapsulated product is spray dried to produce nanoparticles coated with the non-solvent soluble polymer.

11. The method of claim 10 further comprising adding a solution to the nanoparticles coated with non-solvent soluble polymer to produce a suspension.

12. The method of claim 10 further comprising compressing the nanoparticles coated with the non-solvent soluble polymer to produce a solid oral dosage form.

13. The method of claim 1 wherein the agent is dissolved in the solvent.

14. The method of claim 1 wherein the agent is dispersed as solid particles in the solvent.

15. The method of claim 1 wherein the agent is contained in droplets dispersed in the solvent.

16. The method of claim 1 wherein the agent is a liquid.

17. The method of claim 1 wherein the agent is a bioactive agent.

18. The method of claim 17 wherein the bioactive agent is selected from the group consisting of: an amino acid; an analgesic; an anti-anginal; an antibacterial; an anticoagulant; an antifungal; an antihyperlipidemic; an anti-infective; an anti-inflammatory; an antineoplastic; an anti-ulcerative; an antiviral, a bone resorption inhibitor; a cardiovascular agent; a hormone; a peptide; a protein; a hypoglycemic; an immunomodulator; an immunosuppressant; a wound healing agent; and a nucleic acid.

19. The method of claim 1 wherein the nanoencapsulated product consists of particles having an average particle size between 10 nanometers and 10 micrometers.

20. The method of claim 1 wherein the nanoencapsulated product consists of particles having an average particle size between 10 nanometers and 5 micrometers.

21. The method of claim 1 wherein the nanoencapsulated product consists of particles having an average particle size between 10 nanometers and 2 micrometers.

22. The method of claim 1 wherein the nanoencapsulated product consists of particles having an average particle size between 10 nanometers and 1 micrometer.

23. The method of claim 1 wherein a solvent:non-solvent volume ratio is between 1:10 and 1:100.

24. The method of claim 1 wherein a solvent:non-solvent volume ratio is between 1:10 and 1:200.

25. The method of claim 1 wherein the polymer concentration in the solvent phase is between 0.1% and 5% (weight per volume).

26. A method for preparing nanoparticles comprising:
preparing a solution of non-solvent containing a non-solvent soluble polymer and nanoparticles and removing the non-solvent to produce and collect non-solvent soluble polymer coated nanoparticles.

27. The method of claim 26 wherein the non-solvent is selected from the group consisting of: mixtures of isopropyl alcohol and water; mixtures of ethyl alcohol and water; and mixtures of methyl alcohol and water.

28. The method of claim 26 wherein the non-solvent soluble polymer is selected from the group consisting of: polyvinylpyrrolidone; polyethylene glycol; starch; lecithin; and other natural and synthetic water-soluble polymers.

29. The method of claim 26 wherein the nanoparticles further comprise an adhesion promoting agent that promotes adhesion of the polymer-coated nanoparticle to a mucosal surface of a subject.

30. The method of claim 29 wherein the adhesion promoting agent is chosen from the group consisting of: iron oxide, calcium oxide, other metal oxides, fumaric acid anhydride oligimers, poly(fumaric/co-sebacic acid anhydride), and other polyanhydrides, and acid anhydride oligimers.

31. The method of claim 26 wherein the non-solvent soluble polymer is polyvinylpyrrolidone and the non-solvent is a mixture of isopropyl alcohol and water.

32. The method of claim 26 wherein the nanoparticles consists of particles having an average particle size between 10 nanometers and 10 micrometers.

33. The method of claim 26 wherein the nanoparticles consists of particles having an average particle size between 10 nanometers and 5 micrometers.

34. The method of claim 26 wherein the nanoparticles consists of particles having an average particle size between 10 nanometers and 2 micrometers.

35. The method of claim 26 wherein the nanoparticles consists of particles having an average particle size between 10 nanometers and 1 micrometer.

36. The method of claim 26 further comprising preparing a suspension of the nanoparticles.

37. A suspension of nanoencapsulated product comprising a solution of 0.5% to 10% non-solvent soluble polymer and nanoparticles having an average particle size of less than 10 micrometers.

38. The suspension of claim 37 wherein the average particle size of the nanoparticles is less than 1 micrometer.

39. The suspensionof claim 37 wherein the nanoparticles include an agent.

40. A composition comprising nanoparticles having an average particle size of less than 10 micrometers and coated with a non-solvent soluble polymer.

41. The composition of claim 40 wherein the average particle size of the nanoparticles is less than 1 micrometer.

42. The composition of claim 40 wherein the composition is compressed into a solid oral dosage form.

43. The composition of claim 40 wherein the nanoparticles include an agent.

44. A method for delivering an agent to a subject comprising administering to a subject a suspension of claim 39 or a composition of claim 43 to the subject.

45. A method for encapsulating an agent, comprising:
performing phase inversion nanoencapsulation by combining a polymer, an aggregation inhibitor and an agent in an effective amount of a solvent to form a continuous mixture, and introducing the continuous mixture into an effective amount of a non-solvent to cause the spontaneous formation of a nanoencapsulated product.

46. The method of claim 45 wherein the polymer is selected from the group consisting of: polylactic acid, polyglycolic acid, copolymers of lactic and glycolic acid, and other degradable and non-degradable polyesters.

47. The method of claim 45 wherein the polymer concentration in the solvent phase is between 0.1% and 10% (weight per volume).

48. The method of claim 45 wherein the solvent mixture includes an adhesion promoting agent that promotes adhesion of the nanoencapsulated product to a mucosal surface of a subject.

49. The method of claim 48 wherein the adhesion promoting agent is selected from the group consisting of: iron oxide, calcium oxide, other metal oxides, fumaric acid anhydride oligomers, poly(fumaric/co-sebacic acid anhydride), and other polyanhydrides and acid anhydride oligomers.

50. The method of claim 45 wherein the aggregation inhibitor concentration in the solvent is between 0.01% and 10% (weight per volume).

51. The method of claim 45 wherein the aggregation inhibitor is dissolved in the solvent.

52. The method of claim 45 wherein the aggregation inhibitor is dispersed in the solvent.

53. The method of claim 45 wherein the aggregation inhibitor is selected from the group consisting of: poly(vinylpyrrolidone), poly(ethylene glycol), starch, lecithin, modified cellulose and other natural and synthetic water-soluble or insoluble polymers.

54. The method of claim 45 wherein the agent is a liquid.

55. The method of claim 45 wherein the agent is dissolved in the solvent.

56. The method of claim 45 wherein the agent is dispersed as solid particles in the solvent.

57. The method of claim 45 wherein the agent is contained in droplets dispersed in the solvent.

58. The method of claim 45 wherein the agent is a bioactive agent.

59. The method of claim 58 wherein the bioactive agent is selected from the group consisting of: an amino acid, an analgesic, an anti-anginal, an antibacterial, an anticoagulant, an antifungal, an antihyperlipidemic, an anti-infective, an anti-inflammatory, an antineoplastic, an anti-ulcerative, an antiviral, a bone resorption inhibitor, a cardiovascular agent, a hormone, a peptide, a protein, a hypoglycemic, an immunomodulator, an immunosuppressant, a wound healing agent, and a nucleic acid.

60. The method of claim 45 further comprising freezing the mixture of the solvent, the polymer, the aggregation inhibitor, and the agent to form a frozen mixture, drying the frozen mixture, and re-dissolving the dried mixture in a solvent prior to addition to the non-solvent.

61. The method of claim 60 wherein the frozen mixture is dried by vacuum.

62. The method of claim 60 wherein the mixture of the solvent, the polymer, the aggregation inhibitor, and the agent is frozen in liquid nitrogen.

63. The method of claim 45 wherein a solvent:non-solvent volume ratio is between 1:10 and 1:1000.

64. The method of claim 45 wherein a solvent:non-solvent volume ratio is between 1:10 and 1:200.

65. The method of claim 45 wherein the nanoencapsulated product consists of particles having an average particle size between 10 nanometers and 10 micrometers.

66. The method of claim 45 wherein the nanoencapsulated product consists of particles having an average particle size between 10 nanometers and 5 micrometers.

67. The method of claim 45 wherein the nanoencapsulated product consists of particles having an average particle size between 10 nanometers and 2 micrometers.

68. The method of claim 45 wherein the nanoencapsulated product consists of particles having an average particle size between 10 nanometers and 1 micrometer.

69. The method of claim 45 further comprising adding an aggregation inhibitor to the non-solvent.

70. The method of claim 69 wherein the aggregation inhibitor is added to the non-solvent and to the solvent prior to introduction of the continuous mixture into the non-solvent.

71. The method of claim 70 wherein the aggregation inhibitor concentration in the solvent is between 0.01% and 10% (weight per volume) and in the non-solvent is between 0.1% and 20% (weight per volume).

72. The method of claim 69 wherein the aggregation inhibitor is added to the non-solvent prior to introduction of the continuous mixture into the non-solvent.

73. The method of claim 72 wherein the aggregation inhibitor concentration in the non-solvent is between 0.1% and 20% (weight per volume).

74. The method of claim 69 wherein the aggregation inhibitor is added to the non-solvent after introduction of the continuous mixture into the non-solvent.

75. The method of claim 74 wherein the aggregation inhibitor concentration in the solvent is between 0.01% and 10% (weight per volume) and in the non-solvent is between 0.1% and 20% (weight per volume).

76. The method of claim 45 further comprising adding a solution to the nanoencapsulated product to produce a suspension.

77. The method of claim 45 further comprising compressing the nanoencapsulated product to produce a solid oral dosage form.

78. A method for encapsulating an agent, comprising:
performing phase inversion nanoencapsulation by combining a polymer and an agent in an effective amount of a solvent to form a continuous mixture, and introducing the continuous mixture into an effective amount of a non-solvent to cause the spontaneous formation of a nanoencapsulated product, wherein a water-insoluble aggregation inhibitor is added to the non-solvent.

79. The method of claim 78 wherein the polymer is selected from the group consisting of: polylactic acid, polyglycolic acid, copolymers of lactic and glycolic acid, other degradable and non-degradable polyesters, poly(fumaric/co-sebacic acid anhydride), and other polyanhydrides.

80. The method of claim 78 wherein the solvent mixture includes an adhesion promoting agent that promotes adhesion of the nanoencapsulated product to a mucosal surface of the body of a subject.

81. The method of claim 80 wherein the adhesion promoting agent is chosen from the group consisting of: iron oxide, calcium oxide, other metal oxides, fumaric acid anhydride oligomers, poly(fumaric/co-sebacic acid anhydride), and other polyanhydrides and acid anhydride oligomers.

82. The method of claim 78 wherein the water-insoluble aggregation inhibitor is selected from the group consisting of: talc, kaolin, and colloidal silicon dioxide, or any other pharmaceutically acceptable glidant.

83. The method of claim 78 wherein the agent is a bioactive agent.

84. The method of claim 83 wherein the bioactive agent is selected from the group consisting of: an amino acid, an analgesic, an anti-anginal, an antibacterial, an anticoagulant, an antifungal, an antihyperlipidemic, an anti-infective, an anti-inflammatory, an antineoplastic, an anti-ulcerative, an antiviral, a bone resorption inhibitor, a cardiovascular agent, a hormone, a peptide, a protein, a hypoglycemic, an immunomodulator, an immunosuppressant, a wound healing agent, and a nucleic acid.

85. The method of claim 78 wherein the water-insoluble aggregation inhibitor is added to the non-solvent prior to the introduction of the continuous mixture into the non-solvent.

86. The method of claim 78 wherein the water-insoluble aggregation inhibitor is added to the non-solvent after the introduction of the continuous mixture into the non-solvent.

87. The method of claim 78 wherein the concentration of water-insoluble aggregation inhibitor in the non-solvent is between 0.1% and 20% (weight per volume).

88. A nanoencapsulated product prepared according to the methods of any one of claims 45-87.

89. A method for delivering an agent to a subject, comprising administering to a subject a nanoencapsulated product of claim 88, including the agent, to the subject.
